(19) United States
(12) Patent Application Publication
(10) Pub. No.: US 2021/0260589 A1
(43) Pub. Date: Aug. 26, 2021

(54) Method and Apparatus for Measuring Blood Coagulation

(71) Applicant: Emmanuel Mpock, Salida, CA (US)

(72) Inventor: Emmanuel Mpock, Salida, CA (US)

(21) Appl. No.: 17/186,028

(22) Filed: Feb. 26, 2021

Related U.S. Application Data
(60) Provisional application No. 62/981,903, filed on Feb. 26, 2020.

(51) Int. Cl. B01L 3/00; B01L 3/02

(52) U.S. Cl. CPC B01L 3/523; B01L 3/0265; B01L 2200/0621; B01L 2200/0684; B01L 2400/0638; B01L 2300/022; B01L 2300/0838; B01L 2400/0487; B01L 2400/0611; B01L 2200/16

(57) ABSTRACT

An apparatus and methods for performing blood coagulation assays using whole blood or plasma samples in a small portable handheld device.

### FIELD OF INVENTION

The present invention relates to microfluidics systems for performing assays for determining the presence of selected analytes in a sample, in particular, for measuring blood coagulation.

### BACKGROUND

Blood clotting is a hemostatic complex process in which blood coagulation agents are triggered to execute automatically within blood vessels and tissues. The two well-recognized coagulation pathways are the extrinsic or thromboplastin-controlled and the intrinsic or prothrombin/fibrinogen-controlled coagulation pathway. Both pathways result in the production of thrombin, an enzyme which catalyzes the conversion of fibrinogen to fibrin. The soluble plasma protein fibrinogen is converted to fibrin by the action of the enzyme thrombin, resulting in the polymerization of the insoluble fibrin.

Typically, in clinical test labs, blood clotting assays measure the time required for the formation of a fibrin clot. The two most common coagulation tests are Prothrombin Time (PT) and Activated Partial Prothrombin Time (APTT) which are used to measure clotting time to evaluate a patient's hemostatic state or to monitor any chronic or temporary anticoagulant therapy a patient may be undergoing. Other types of coagulation assays include partial thromboplastin time (PTT), activated partial thromboplastin time (APTT), fibrinogen assay, thrombin clotting time (TCT), activated clotting time (ACT), among others.

The PT test is used to assess the extrinsic and common pathway of clotting systems and for monitoring long term anticoagulant therapy. A common medication for long term anticoagulant therapy is sodium warfarin isopropanol clathrate, generally known by the brand name COUMADIN™. The amount of Warfarin in the blood is directly related to the clotting time and as such the clotting time can be regulated and tracked by taking PT measurements of a patient with the dosage implemented as deemed appropriate.

The second routinely used assay, APTT test, is widely used for monitoring Heparin therapy for screening deficiencies of clotting factors included in the intrinsic and common coagulation system.

There are various methods and systems currently in the market used for performing coagulation assays in the laboratories and some clinics. There are many disadvantages in the use of some of these methods. The laboratory instruments are relatively large because of the complex technology used, they are expensive, and designed for use by trained personnel due to their complexity. In addition, large blood samples volumes are also usually required and to reduce cost, testing is done in large batches collected over days, resulting in long delays before results are obtained. The blood samples for larger equipment are generally collected through venous blood draws, processed and refrigerated or transported to wherever the testing has to be done. There are a few used at the point of care settings for rapid testing but they are also plagued with multiple challenges. These challenges include accuracy, sensitivity, reproducibility and usability from attempts in trying to measure clot formation indirectly and these challenges are compounded for systems designed for home use. Thus, there is a need for assay systems for detecting and determining the time for blood coagulation that are small and portable, accurate and sensitive, require very small sample volumes, and are very simple to use even at home by the elderly, and are inexpensive. The device of the invention is small, inexpensive, accurate, sensitive, robust and easy to use.

### SUMMARY

The present invention provides apparatus and methods for performing assays for determining the time required for a blood sample to coagulate. The device of the invention is small, inexpensive, accurate, sensitive, robust and easy to use. The apparatus comprises of a cartridge coated with one or more wet or dry clotting agents. A drop of whole blood or plasma is automatically aspirated into the cartridge when brought into contact with the sample entry point, wherein the sample reacts with the clotting agents. During the reaction, mechanically and electrically pulsed fibrin probes blanket the reacting sample until the blood sample begins the clotting process and the clot is easily detected within a fraction of a second from the onset of its formation. The blood clotting process forms fibrin stands that are directly detected, electrically, mechanically and or optically, at the onset of the fibrin polymerization. The clotting time is the total time from the sample making contact with the chemistry in the cartridge, to the time at which the onset of fibrin polymerization is detected and registered by the probes.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates a perspective view of the disposable cartridge with a fibrin probe system, optic module and support electrical circuitry.

FIG. 2a illustrates a perspective view of the reader device.

FIG. 2b FIG. 2a illustrates a perspective view of the reader device.

FIG. 3 FIG. 2a illustrates a cut-away view of the reader device.

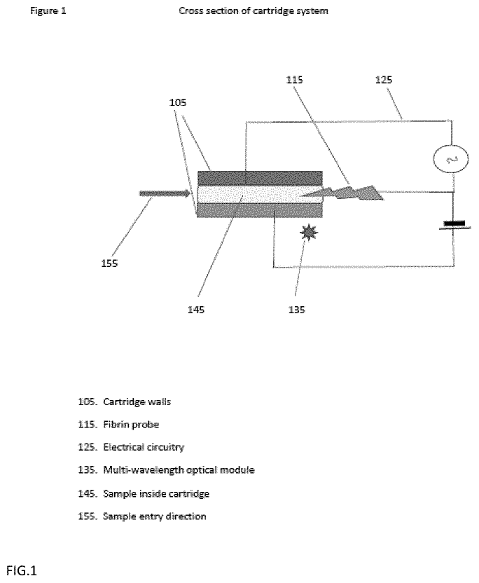

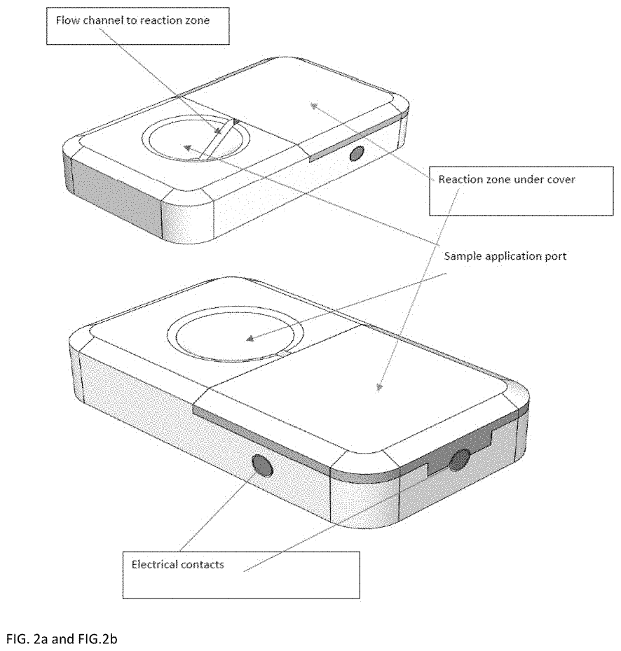

### DETAILED DESCRIPTION

All publications, patents and patent applications cited herein, whether supra or infra, are hereby incorporated by reference in their entirety.

As used herein, the term “subject” encompasses humans, nonhumans or any organism with clottable life supporting liquid matrix capable of clotting reaction with the precipitation of the equivalent of a fibrin polymer-like material.

The present invention provides apparatus and methods for performing blood coagulation assays using whole blood or plasma samples in a small portable handheld device. The apparatus comprises of a small cartridge coated with one or more clotting agents and armed with an electrical fibrin seeking probe. A very small drop of blood or plasma is automatically aspirated into the cartridge when brought into contact with the sample entry point, wherein the sample reacts with the clotting agents. The reaction mixture can be subjected to single or multiple electrical fibrin strand seekers in the tiny capillary system and may or may not include an optical filtration mechanism for visualization of the onset of fibrin polymerization. The blood clotting process forms microscopic fibrin strands that are quickly and physically detected as the polymerization process begins. The clotting time is the total time from the sample entering the reaction chambers to the time at which the polymerization begins, and can be measured electrically and/or optically.

The invention pertains to methods and microfluidic apparatus for measuring blood coagulation time. A typical microfluidic apparatus is illustrated in the figures. The apparatus comprises a solid enclosure or cartridge 105 that may be coated with one or more clotting agents such as tissue factor or other clotting agents. A drop of blood or plasma is automatically aspirated through the proximal end of the disposable apparatus 155 flowing into the capillary opening 145 when brought into contact with the disposable apparatus, and upon coming into contact with the clotting agents, the clotting process is triggered. The fibrin seeking probe 115 probes the enclosure 145 seeking fibrin fiber strands until detected by the probe system. The clotting time is the total time from the sample entering the cartridge 105 to the time at which the electrical, mechanical or optical pulse changes when the fibrin fibers are detected by the probe.

The cartridge 105 can be any shape, preferably (but not limited to) a tubular with a distal and proximal end in and enclosure and can be made from a suitable material that is selected on its properties, such as good thermal conductivity, can be clear or opaque, can be constructed to provide a rigid support for the probe systems and non-absorptive to the liquid sample being tested. For this purpose, suitable plastics include those with high free surface energies and low water sorption, including nonrusting metals, synthetic and non-synthetic plastic media like stainless steel, PETG, Mylar®, polycarbonate, polyvinyl chloride, polystyrene, SAN, acrylonitrile-butadiene-styrene (ABS), among others. Alternatively and equivalently, a commercially-available molded cartridge can be used in the practice of the invention.

The disposable apparatus or cartridge 105 can as well as the probe can be coated with one or more clotting agents, such as tissue factors, diatomaceous earth, etc. The cartridge lumen is wide enough to grant access to the micro fibrin seeking probes.

For the present invention, the blood sample is naturally obtained from a patient by means such as venipuncture or a finger prick and the drop can be brought directly in touch with the disposable apparatus or cartridge 105. In one aspect of the invention, the sample of blood obtained from the patient can be used with or without additional manipulation. Since the probes seek just fibrin, the sample in use can be whole blood or just plasma.

The clotting assays of the present invention include prothrombin time (PT), partial thromboplastin time (PTT), activated partial thromboplastin time (APTT), thrombin clotting time (TCT), fibrinogen, heparin management test (HMT), protamine response time (PRT), heparin response time (HRT), low molecular weight heparin (LMWH), low range heparin management test (LHMT), and ecarin clotting time (ECT), with the reagents for each of these tests as described in the art. The reagents for one or more the assays are simply coated in the cartridge 105.

For example, if the clotting assay is PT, any source of thromboplastin can be used, either recombinant sources or that obtained from rabbit brains and placentas. Powdered bovine brain, powdered ox brain, powdered human brain, powdered thromboplastin produced by recombinant DNA technology, and powdered human placenta can be prepared by well known art methods. These are all commercially available. For example, thromboplastin can be purchased from a commercial source or it can be extracted from human placenta, rabbit brain, bovine brain, ox brain, and human brain. Thromboplastin produced by recombinant DNA technology can also be used.

In another example, the clotting assay can be the Protein C activator assay, and the methods and apparatus of the present invention are illustrated using the Protein C activator assay. This assay uses a reagent comprising an initiator of the intrinsic coagulation pathway or Factor X, a Protein C activator (such as thrombomodulin, Protac™, other catalytic or stoichiometric activators), calcium ions, one or more metal compounds that interact with calcium binding sites in the coagulation cascade, preferably one or more lanthanide compounds, and optionally bulkers and/or stabilizers.

In one aspect of the invention, a cycling electrical circuit is irreversibly disrupted by the polymerizing fibrin as it is snagged by the fibrin probe sweeping through the sample and this signals the onset of clot formation.

Typically, the fibrin probe 115 system could be made of conducting or nonconducting material and is part of the electrical circuit and or mechanical system. The cyclical probing of the fibrin probe 115 is executed within the disposable apparatus lumen 145 inside the sample reagent mixture resulting in a very clear and precise electrical wave pattern. When the fibrin polymerization begins (at the coagulation point) the fibrin mesh in the clot wraps around the probe bridging electrical contacts that are isolated prior to the clot formation thus irreversibly shorting the electrical system.

In another aspect of this invention, the same cartridge made using a combination of a transparent and opaque matrices or all transparent matrices, is used in conjunction with a multi wavelength optical module whereby the coagulation process can be tracked transmissively or reflectively. The sample is tracked from the time of entry to the onset of clot formation, and this time is determined as the clotting time. In this aspect of this invention, a plurality of wavelengths is needed to accurately capture the point at which the fibrin polymerization occurs. While this may be relatively easy to determine in a plasma sample, the presence of platelets, red and white blood cells make this polymerization just a little less obvious to detect directly with a single wavelength, when using a whole blood sample. Two wavelengths in combination serve as reference points while at least a third, used as an additive or filtering element in real time, will serve to correctly determine and amplify the polymerization point in a normal blood sample that will sustain normal bodily functions.

In another aspect of this invention, the optical version can be coupled to work in tandem with the electrical version in enhancing the sensitivity of the clot detection.

In another aspect of this invention, the clot formation can be detected by a change in the capacitance as one of many of the electrical and physical changes that are measurable during clot formation.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

### Advantages of the Invention

The device of the invention is small, inexpensive, accurate, sensitive, robust and easy to use. In size and cost it is similar to a home use glucose monitors used by diabetics. It will be used for home testing, testing at a doctor's office, and testing in a hospital setting at the point of care during cardiopulmonary bypass, cardiac catheterization, angioplasty, haemodialysis and other procedures where heparin monitoring with accurate ACT results is frequently needed. Current instruments used for these procedures are expensive and large, typically being table top systems that cost thousands of dollars and requiring special training. A typical system will cost more than $5000, and have approximate minimum dimensions of 12×12×10 inches, being a minimum volume of 1440 inches cubed.

Commercial accessibility. Another advantage of the invention is that it is inexpensive to make. In size and cost it is similar to a home use glucose monitors used by diabetics. After initial capital investment, the entire production cost in 2020 prices, taking into account the cost of all components and the cost of assembly, should be no more than about $20.

Ease of use. Another very important advantage of the invention is that it is simple to use. No specialized training is required and a person with no training can easily perform the test themselves by procuring a very small volume of blood from a finger-prick. Unlike some other blood/serum assays, where accurate results cannot easily be obtained from a small volume of whole blood, the coagulation assay works well in a small volume such as the volume of the sample chamber of the present invention. The system is designed to detect sample volume and is almost autonomous in its operation, requiring only the insertion of the cartridge and application of a drop of sample. No other activity is required to run a test.

Size. Size is a very important advantage of this invention. No other device with this footprint and capability exists in the market. The size of the reader device into which the cartridge is placed is equivalent to a cell phone. An exemplary device has approximate maximum dimensions of between 7×6×2 inches and 4×3×1 inches, for example a preferred example of the deceive would have approximate maximum dimensions of 5×4×1 inches or 5×4×0.75 inches of or 5×4×0.5. The size of the device will be no larger than 7×6×2 inches, and the volume will be no larger than 84 inches cubed. A preferred version will have a volume of no more than 20 inches cubed. The small size of the device makes it easily portable and convenient for home use. The maximum dimensions of the cartridge that is placed in the reader is similar to a credit card and will be no larger than 7×6×2 inches, and the volume will be no larger than 84 inches cubed 4×3×0.25 inches (3 inches cubed), or preferable lo larger than 3.5×2.5×0.1 inches (0.875 inches cubed).

Another very important advantage of the invention is the sensitivity of clot detection. The present invention detects clot formation directly at the very onset of fibrin formation, ensuring high device to device reproducibility and very high accuracy using either whole blood or plasma. Other devices need to wait for an accumulation of fibrin to reach a detectable threshold which in some cases could take minutes for unstable clots and those with low fibrinogen levels. This is even more challenging for systems with optical detection methods that rely on change in viscosity of the sample in the presence of red blood cells.

Reagent type. The system is designed to function with a liquid or dried reagent chemistry, as needed. Its dry chemistry format eliminates dilution of the blood sample, requiring just a drop of blood directly from the finger or syringe in its self-volume metering cartridge. No pipetting of premeasured volumes needed.

Homogenization. The clot detection probe of the invention offers a homogenizing mechanism to ensure optimal reagent and sample uniformity and therefore a uniform clot formation with easy detection.

Embodiments of the invention include the following:

A disposable cartridge adapted to be inserted into and used with a matching reader device, the cartridge comprising walls defining or containing therewithin a capillary tube having a sample application port at the proximal end, and a reaction chamber at the distal end, wherein the reaction chamber is adapted to hold a sample volume, wherein the reaction chamber contains a reagent in contact with the reaction chamber, and wherein the disposable cartridge further comprises a clot-seeking probe, which probe is electrically conductive and is adapted to move into and out of the sample volume under the influence of a magnetic field, and wherein, in use, the probe moves into and out of the sample volume intermittently, thereby providing intermittent physical contact between the sample volume and the probe, and wherein the probe is electrically connected as part of a circuit that is further in contact with the reaction chamber, such that when the cartridge is placed within the matching reader, and the probe is in contact with the reaction volume, either directly or through clot material which is in contact with both the reaction volume and the probe, an electrical current flows through the electrical circuit, which current may be detected and indicated to a user by the matching reader.

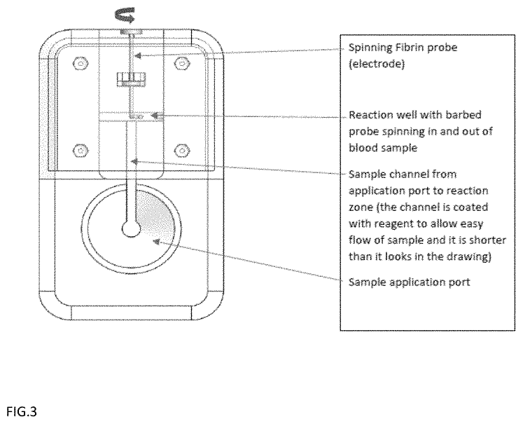

A disposable cartridge is one that is designed to be disposed of after one use. A matching reader device is a machine into which the cartridge is designed to fit, having electrical connections inside that match the circuitry of the cartridge, such that a continuous circuit is formed when the probe is in contact with a sample either directly or via a clot. The capillary tube is any tube capable of capillary action using a blood or serum sample, and will generally be made of glass, ceramic or metal, but may be made of other materials and may be defined by the walls of the device. The reaction chamber is defined as the volume in which a reaction takes place. It may be a widening at the end of the capillary or it may be a part of the contiguous capillary, or it may simply be defined as the capillary itself. It is a functional definition and is not limited to any specific shape or location or volume. A clot-seeking probe is again a functional definition. It is a probe made of any electrically conductive material, and its intended purpose is to contact a clot material in a reaction chamber if such a clot material exists. It can be of any shape and size commensurate with its function. For example in FIG. 3 it is hooked or barbed and is disposed more or less at a right angle on the end of a rotating shaft, such that it dips into and out of the reaction volume. But it may also be a straight, possibly hooked or barbed at one end so as to trap/pick up/attach the clot material. A clot is defined as any fibrous, semi-solid or solid material, so long as it has greater viscosity than the liquid from which it is precipitating.

The probing frequency is adjustable based on the assay being performed.

The cartridge may be adapted to perform an assay selected from the group consisting of prothrombin time (PT), partial thromboplastin time (PTT), activated partial thromboplastin time (APTT), thrombin clotting time (TCT), fibrinogen, heparin management test (HMT), protamine response time (PRT), heparin response time (HRT), low molecular weight heparin (LMWH), low range heparin management test (LHMT), ecarin clotting time (ECT), and combinations thereof.

In various embodiments, the cartridge walls may comprise a substance selected from the group consisting of plastic, glass, nylon, metal, and combinations thereof. The reaction chamber is formed from the distal end of the capillary tube. The device may be capable of detecting clot formation directly at the very onset of fibrin formation. The probe maybe ferromagnetic and it may be made of stainless steel and may be coated with one or more clotting agents. The capillary tube can be made of glass.

The reaction chamber can contain a solid or liquid reagent and the solid reagent may be coated onto the walls of the reaction chamber. The reagent may be an initiator of the intrinsic coagulation pathway and may be selected from the group consisting of Factor X, a Protein C activator, other catalytic or stoichiometric activators, calcium ions, metal compounds and lanthanide compounds.

The cartridge may further comprise a wavelength optical module comprising a polychromatic emitter and detector.

The onset of clotting can be detected electrically as a function of impedance, capacitance, optical density, turbidity and general electrical conductivity.

A sample may be collected from the sample source automatically when brought into contact with the sample entry port of the cartridge. Whole blood or plasma can be used as the sample. A sample detection electrode may be built into the capillary tube to electrically detect the presence of sufficient sample volume to initiate the test.

I claim:

1. A disposable cartridge adapted to be inserted into and used with a matching reader device, the cartridge comprising walls defining or containing there within a capillary tube having a sample application port at the proximal end, and a reaction chamber at the distal end, wherein the reaction chamber is adapted to hold a sample volume, wherein the reaction chamber contains a reagent in contact with the reaction chamber, and wherein the disposable cartridge further comprises a clot-seeking probe, which probe is electrically conductive and is adapted to move into and out of the sample volume under the influence of a magnetic field, and wherein, in use, the probe moves into and out of the sample volume intermittently, thereby providing intermittent physical contact between the sample and the probe, and wherein the probe is electrically connected as part of a circuit that is further in contact with the reaction chamber, such that when the cartridge is placed within the matching reader, and the probe is in contact with the reaction volume, either directly or through clot material which is in contact with both the reaction volume and the probe, an electrical current flows through the electrical circuit, which current may be detected by the reader.

2. The cartridge of claim 1, wherein the cartridge walls comprise a substance selected from the group consisting of plastic, glass, nylon, metal, and combinations thereof.

3. The cartridge of claim 1, wherein the reaction chamber is formed from the distal end of the capillary tube.

4. The cartridge of claim 1, capable of detecting clot formation directly at the very onset of fibrin formation.

5. The cartridge of claim 1, wherein the probe is ferromagnetic.

6. The cartridge of claim 1, wherein the probe is made of stainless steel.

7. The cartridge of claim 1, wherein the probe is coated with one or more clotting agents.

8. The cartridge of claim 1, wherein the capillary tube is made of glass.

9. The cartridge of claim 1, wherein the reaction chamber contains a solid reagent.

10. The cartridge of claim 1, wherein the reaction chamber contains a solid reagent that is coated onto the walls of the reaction chamber.

11. The cartridge of claim 1, wherein the reaction chamber contains a liquid reagent.

12. The cartridge of claim 1, wherein the reaction chamber contains a reagent that is an initiator of the intrinsic coagulation pathway.

13. The cartridge of claim 1, wherein the reaction chamber contains a reagent selected from the group consisting of Factor X, a Protein C activator, other catalytic or stoichiometric activators, calcium ions, metal compounds and lanthanide compounds.

14. The cartridge of claim 1 further comprising a wavelength optical module comprising a polychromatic emitter and detector.

15. The cartridge of claim 1, adapted to perform an assay selected from the group consisting of prothrombin time (PT), partial thromboplastin time (PTT), activated partial thromboplastin time (APTT), thrombin clotting time (TCT), fibrinogen, heparin management test (HMT), protamine response time (PRT), heparin response time (HRT), low molecular weight heparin (LMWH), low range heparin management test (LHMT), ecarin clotting time (ECT), and combinations thereof.

16. The cartridge of claim 1 wherein the onset of clotting can be detected electrically as a function of impedance, capacitance, optical density, turbidity and general electrical conductivity

17. The cartridge of claim 1 wherein the sample is collected from the sample source automatically when brought into contact with the sample entry port of the cartridge

18. The cartridge of claim 1 wherein whole blood or plasma can be used as the sample.

19. The cartridge of claim 1 wherein a sample detection electrode is built into the capillary tube to electrically detect the presence of sufficient sample volume to initiate the test.

20. The cartridge of claim 1 wherein the probing frequency is adjustable based on the assay being performed.
